(19) United States
(12) Patent Application Publication
(10) Pub. No.: US 2025/0022190 A1
(43) Pub. Date: Jan. 16, 2025

(54) INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventor: KOHTARO UMEZAWA, Tokyo (JP)

(21) Appl. No.: 18/767,068

(22) Filed: Jul. 9, 2024

(30) Foreign Application Priority Data
Jul. 10, 2023 (JP) 2023-113002
Apr. 19, 2024 (JP) 2024-068245

(51) Int. Cl. G06T 11/00; G06T 7/00; G06T 7/11

(52) U.S. Cl. CPC G06T 11/008; G06T 7/0012; G06T 7/11; G06T 2207/20104; G06T 2207/20132; G06T 2207/30008; G06T 2207/30101; G06T 2211/408; G06T 2211/464; G06V 2201/031

(57) ABSTRACT

An information processing apparatus includes an image acquisition unit configured to acquire material decomposition image data based on detection signals related to X-ray photons transmitted through a subject in a plurality of respective energy bands, a region of interest acquisition unit configured to acquire a region of interest in the material decomposition image data, a data processing unit configured to generate partial image data of the material decomposition image data corresponding to the region of interest, and a storage unit configured to store the partial image data of the material decomposition image data.

### BACKGROUND OF THE DISCLOSURE

### Field of the Disclosure

The present disclosure relates to an information processing apparatus, an information processing method, and a storage medium for managing image data acquired from a medical image capturing apparatus.

### Description of the Related Art

Dual-energy computed tomography (CT) devices emit X-rays with two different types of energy separately. Differences in luminance values between the obtained respective images are used to generate material decomposition image data having voxel values corresponding to the densities of a plurality of materials expected to be present in a subject.

X-ray CT devices generate energy band-specific material decomposition image data on each of a plurality of materials using detection signals based on the numbers of X-ray photons transmitted through a subject in the respective energy bands. Japanese Patent Application Laid-Open No. 2019-72082 discusses a technique where an X-ray CT device generates combined image data on an image into which the densities of a plurality of materials are combined from a plurality of pieces of material decomposition image data.

According to Japanese Patent Application Laid-Open No. 2019-72082, for example, a plurality of pieces of material decomposition image data is used to generate combined image data, and the plurality of pieces of image data is stored in a storage unit of an information processing apparatus.

### SUMMARY OF THE DISCLOSURE

The present disclosure is directed to reducing the data amount of image data to be stored in a storage unit of an information processing apparatus. The issue that is addressed by exemplary embodiments disclosed in the specification and drawings is not limited to the foregoing. Other issues that are associated with the effects of configurations set forth in the exemplary embodiments described below can also be considered as issues addressed by exemplary embodiments.

According to an aspect of the present disclosure, an information processing apparatus includes an image acquisition unit configured to acquire material decomposition image data based on detection signals related to X-ray photons transmitted through a subject in a plurality of respective energy bands, a region of interest acquisition unit configured to acquire a region of interest in the material decomposition image data, a data processing unit configured to generate partial image data of the material decomposition image data corresponding to the region of interest, and a storage unit configured to store the partial image data of the material decomposition image data.

Further features of the present disclosure will become apparent from the following description of exemplary embodiments with reference to the attached drawings.

### BRIEF DESCRIPTION OF THE DRAWINGS

FIG. 1 is a diagram illustrating an example of a configuration of a medical image capturing apparatus (X-ray computed tomography [CT] device) according to the present disclosure.

FIG. 2 is a diagram illustrating an example of a configuration of an information processing apparatus according to the present disclosure.

FIG. 3 is a diagram illustrating an example of a configuration of the information processing apparatus according to the present disclosure.

FIG. 4 is a flowchart illustrating an example of an operation of the information processing apparatus according to the present disclosure.

FIGS. 5A and 5B are diagrams illustrating acquisition of region of interest masks according to the present disclosure.

FIGS. 6A and 6B are diagrams illustrating acquisition of a combined region of interest mask according to the present disclosure.

FIGS. 7A and 7B are diagrams illustrating acquisition of a combined region of interest mask according to the present disclosure.

FIGS. 8A and 8B are diagrams illustrating acquisition of region of interest masks according to the present disclosure.

FIGS. 9A to 9C are diagrams illustrating an example of data reduction processing according to the present disclosure.

FIGS. 10A to 10C are diagrams illustrating an example of the data reduction processing according to the present disclosure.

FIGS. 11A and 11B are diagrams illustrating data to be stored in a storage unit according to the present disclosure.

FIGS. 12A and 12B are diagrams illustrating data to be stored in the storage unit according to the present disclosure.

FIG. 13 is a flowchart illustrating an example of an operation of an information processing apparatus according to the present disclosure.

FIG. 14 is a diagram illustrating a display mode of a display device according to the present disclosure.

### DESCRIPTION OF THE EMBODIMENTS

Hereinafter, an information processing apparatus, an information processing method, and a storage medium storing a program according to an exemplary embodiment will be described with reference to the drawings. The constituents denoted by the same reference numerals perform the same operations, and the redundant description will be omitted as appropriate. Hereinafter, an exemplary embodiments will be described with reference to the drawings.

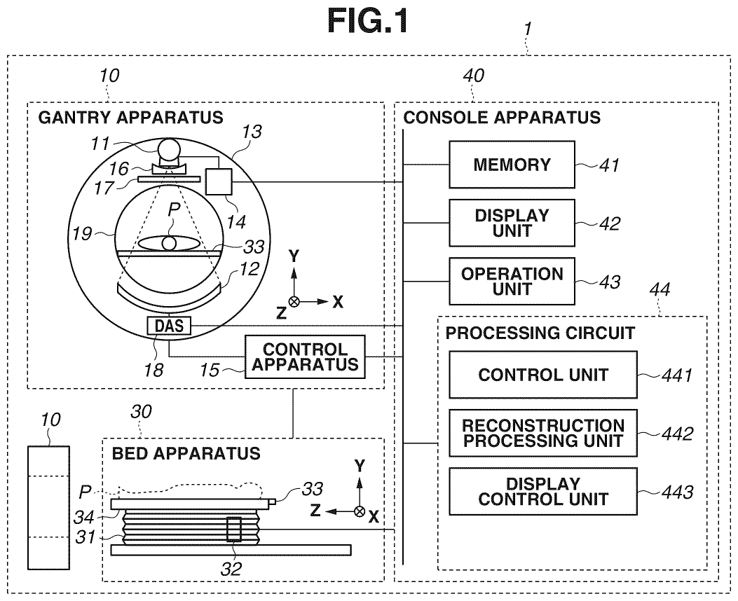

A first exemplary embodiment of the present disclosure will be described with reference to FIG. 1 to FIGS. 10A to 10C. An example of a configuration of a medical image capturing apparatus (X-ray computed tomography [CT] device) according to the first exemplary embodiment will be described with reference to FIG. 1. A medical image capturing apparatus 1 illustrated in FIG. 1 includes a gantry apparatus (gantry) 10, a bed apparatus 30, and a console apparatus 40. In the present exemplary embodiment, the longitudinal direction of the rotation axis of a rotating frame 13 in a non-tilted state or a top plate 33 of the bed apparatus 30 is defined as a Z-axis direction. An axial direction orthogonal to the Z-axis direction and parallel to a floor surface is defined as an X-axis direction. An axial direction orthogonal to the Z-axis direction and perpendicular to the floor surface is defined as a Y-axis direction.

For example, the gantry apparatus 10 and the bed apparatus 30 are installed in a CT scan room. The console apparatus 40 is installed in a control room next to the CT scan room. However, the console apparatus 40 does not necessarily need to be installed in the control room. For example, the console apparatus 40 may be installed in the same room as the gantry apparatus 10 and the bed apparatus 30. In any case, the gantry apparatus 10, the bed apparatus 30, and the console apparatus 40 are communicably connected to each other in a wired or wireless manner.

The gantry apparatus 10 is a scan device including a configuration for capturing an X-ray CT image of a subject P. The gantry apparatus 10 includes an X-ray tube 11, a detector 12, the rotating frame 13, an X-ray high voltage apparatus 14, a control apparatus 15, a wedge 16, a collimator 17, and a data acquisition apparatus 18 (hereinafter, also referred to as a data acquisition system [DAS]).

For the convenience of description, only some of the components will be described below.

The X-ray tube 11 is a vacuum tube that generates X-rays by irradiating an anode (target) with thermionic electrons from a cathode (filament) through application of a high voltage and supply of a filament current from the X-ray high voltage apparatus 14. Specifically, thermionic electrons impinge on the target to generate X-rays. An example of the X-ray tube 11 is a rotating anode X-ray tube that generates X-rays by irradiating a rotating anode with thermionic electrons. The X-rays generated by the X-ray tube 11 are shaped into a cone beam through the collimator 17, for example, and emitted onto the subject P. The X-ray tube 11 is an example of an X-ray generation unit.

The detector 12 detects the X-rays emitted from the X-ray tube 11 and transmitted through the subject P, and outputs a signal corresponding to the amount of X-rays to the DAS 18. For example, the detector 12 includes a plurality of X-ray detection element rows where a plurality of X-ray detection elements is arranged in a channel direction along an arc about the focal point of the X-ray tube 11. For example, the detector 12 has a row structure where the plurality of X-ray detection element rows including the plurality of X-ray detection elements arranged in the channel direction is arranged in a slice direction (row direction). The detector 12 is a photon-counting detector. By counting X-ray photons transmitted through the subject P with the detector 12, a reconstruction processing unit 442 can reconstruct high signal-to-noise (S/N) ratio integration image data (CT image data).

The functions of the detector 12 can be classified into an energy integration function and a photon counting function depending on the measurement method of the DAS 18 that is used for the converted signal. The energy integration function measures the total amount of X-ray energy transmitted in a specific time by integrating the energy of the X-rays transmitted through the subject P over the specific time. The photon counting function measures the numbers of X-ray photons included in the X-rays transmitted through the subject P in a plurality of respective energy bands (also referred to as energy bins, or simply bins) separately. This enables material decomposition based on captured data acquired in the respective energy bands. As employed herein, an imaging mode involving material decomposition will be referred to as a material separation mode (material decomposition image data mode). An imaging mode not involving material decomposition will be referred to as a material non-decomposition mode (integration image data mode).

The rotating frame 13 rotatably supports the X-ray generation unit and the X-ray detection unit (detector 12) about a rotation axis Z. Specifically, the rotating frame 13 is an annular frame that supports the X-ray tube 11 and the detector 12 in a manner such that the X-ray tube 11 and the detector 12 face each other, and rotates the X-ray tube 11 and the detector 12 under the control of the control apparatus 15. The rotating frame 13 is rotatably supported by a fixed frame (not illustrated) formed of metal such as aluminum. More specifically, the rotating frame 13 is connected to the rim of the fixed frame via bearings. The rotating frame 13 is powered by a driving mechanism of the control apparatus 15 and rotates about the rotation axis Z at a constant angular velocity.

The rotating frame 13 further includes and supports the X-ray high voltage apparatus 14 and the DAS 18 in addition to the X-ray tube 11 and the detector 12. Such a rotating frame 13 is accommodated in a substantially cylindrical housing that has an opening (bore) 19 forming an imaging space. The opening 19 roughly matches a field of view (FOV). The center axis of the opening 19 agrees with the rotation axis Z of the rotating frame 13. Captured data generated by the DAS 18 is transmitted from a transmitter (not illustrated) including a light-emitting diode (LED) to a receiver (not illustrated) including a photodiode, for example. Such transmission processing is performed on the photodiode installed on a non-rotating portion (fixed frame) of the gantry apparatus 10 by optical communication. The captured data is then transferred from the receiver to the console apparatus 40.

The method for transmitting the captured data from the rotating frame 13 to the non-rotating portion of the gantry apparatus 10 is not limited to the optical communication. Any contactless data transmission method may be employed.

The X-ray high voltage apparatus 14 includes electric circuits, such as a transformer and a rectifier. The X-ray high voltage apparatus 14 further includes a high voltage generation apparatus having a function of generating a high voltage to be applied to the X-ray tube 11 and a filament current to be supplied to the X-ray tube 11. The X-ray high voltage apparatus 14 further includes an X-ray control apparatus that controls an output voltage based on the X-rays to be emitted from the X-ray tube 11.

The control apparatus 15 includes a processing circuit including a central processing unit (CPU), and a driving mechanism, such as a motor and an actuator. The processing circuit includes a processor, such as the CPU and a microprocessing unit (MPU), and a memory, such as a read-only memory (ROM) and a random access memory (RAM), as its hardware resources. The control apparatus 15 controls the X-ray high voltage apparatus 14 and the DAS 18 based on instructions from the console apparatus 40. The processor performs the foregoing control by reading a program stored in the memory and executing the program.

The wedge 16 is a filter for adjustment of the amount of X-rays emitted from the X-ray tube 11.

Specifically, the wedge 16 is a filter for transmission and attenuation of the X-rays emitted from the X-ray tube 11 so that the X-rays emitted from the X-ray tube 11 onto the subject P have a predetermined distribution. For example, the wedge 16 (wedge filter or bow-tie filter) is a filter formed by machining an aluminum plate to a predetermined target angle and a predetermined thickness.

The collimator 17 is lead plates narrowing the irradiation range of the X-rays transmitted through the wedge 16, and has slits formed by combining the plurality of lead plates. The collimator 17 may be referred to as an X-ray diaphragm.

The DAS 18 generates digital data (also referred to as captured data) indicating the count of X-ray photons detected by the detector 12 in each of a plurality of energy bands. The captured data is a set of count values identified by channel numbers and row numbers of the source X-ray detection elements, a view number indicating the collected view (projection angle), and an energy bin number. The captured data is transferred to the console apparatus 40.

The bed apparatus 30 is an apparatus for bearing and moving the subject P to be scanned. The bed apparatus 30 includes a base 31, a bed driving apparatus 32, the top plate 33, and a support frame 34. The base 31 is a housing for vertically movably supporting the support frame 34.

The bed driving apparatus 32 is a motor or actuator for moving the top plate 33, on which the subject P is placed, in the longitudinal direction of the top plate 33. The top plate 33 disposed on the top surface of the support frame 34 is a plate on which the subject P is placed. The bed driving apparatus 32 may move the support frame 34 in the longitudinal direction of the top plate 33 in addition to the top plate 33.

The console apparatus 40 includes a memory 41, a display unit 42, an operation unit 43, and a processing circuit 44. Data communication between the memory 41, the display unit 42, the operation unit 43, and the processing circuit 44 is performed via a bus. While the console apparatus 40 is described as a separate unit from the gantry apparatus 10, the console apparatus 40 or some of the components of the console apparatus 40 may be included in the gantry apparatus 10.

The memory 41 is a storage device for storing various types of information, such as a hard disk drive (HDD), a solid stage drive (SSD), and an integrated circuit storage device.

For example, the memory 41 stores captured data, integration image data, and material decomposition image data. Aside from the HDD and SSD, the memory 41 may be a drive for reading and writing various types of information from/to a portable storage medium, such as a Compact Disc (CD), a Digital Versatile Disc (DVD), and a flash memory, or a semiconductor memory element, such as a RAM. The memory 41 may be included in the medical image capturing apparatus 1, or in a network-connected external storage device. The memory 41 stores a control program according to the exemplary embodiment.

The display unit 42 displays various types of information. For example, the display unit 42 outputs image data (CT image data) generated by the processing circuit 44, and a graphical user interface (GUI) for accepting various operations from the user. For example, a liquid crystal display (LCD), a cathode ray tube (CRT) display, an organic electroluminescence display (OELD), a plasma display, or any other display can be appropriately used as the display unit 42. The display unit 42 may be included in the gantry apparatus 10. The display unit 42 may be a desktop display unit, or a tablet terminal capable of wireless communication with the main body of the console apparatus 40.

The operation unit 43 accepts various input operations from the user, converts the accepted input operations into electrical signals, and outputs the electrical signals to the processing circuit 44. For example, the operation unit 43 accepts, from the user, collection conditions in collecting captured data, reconstruction conditions in reconstructing CT image data, and image processing conditions in generating a postprocessed image from the CT image data. For example, a mouse, a keyboard, a trackball, a switch, a button, a joystick, a touch pad, and/or a touchscreen display unit can be appropriately used as the operation unit 43. In the exemplary embodiment, the operation unit 43 is not limited to ones having physical operation parts like a mouse, a keyboard, a trackball, a switch, a button, a joystick, a touch pad, and a touchscreen display unit. Examples of the operation unit 43 may include an electrical signal processing circuit that receives electrical signals corresponding to input operations from an external input device disposed separate from the console apparatus 40 and outputs the electrical signals to the processing circuit 44. The operation unit 43 may be included in the gantry apparatus 10. The operation unit 43 may be a tablet terminal capable of wireless communication with the main body of the console apparatus 40.

The processing circuit 44 controls entire operation of the medical image capturing apparatus 1 based on the electrical signals of the input operations output from the operation unit 43. For example, the processing circuit 44 includes a processor, such as a CPU, an MPU, and a graphics processing unit (GPU), and a memory, such as a ROM and a RAM, as its hardware resources. The processing circuit 44 implements a control unit 441, the reconstruction processing unit 442, and a display control unit 443 by using a processor that executes programs loaded in a memory. The functions (control unit 441, reconstruction processing unit 442, and display control unit 443) are not limited to the implementation by a single processing circuit. A plurality of independent processors may be combined to constitute the processing circuit 44, and the processors may implement the functions by executing programs.

The control unit 441 controls the functions of the processing circuit 44 based on the input operations accepted from the user via the operation unit 43. Specifically, the control unit 441 reads the control program stored in the memory 41, loads the control program into the memory in the processing circuit 44, and controls various parts of the medical image capturing apparatus 1 based on the loaded control program. The control unit 441 also generates imaging data by applying preprocessing such as logarithmic conversion processing, offset correction processing, channel-to-channel sensitivity correction processing, and beam hardening correction to the captured data output from the DAS 18.

The reconstruction processing unit 442 has a function of generating CT image data based on the captured data output from the detector 12 (DAS 18). The reconstruction processing unit 442 reconstructs CT image data by performing back projection processing on captured data stored in the memory 41, for example. Examples of the back projection processing include filtered back projection (FBP)-based back projection processing. The reconstruction processing unit 442 may perform the reconstruction processing by iteration, for example. The reconstruction processing unit 442 also generates new CT image data by performing various types of image processing on CT image data. The reconstruction processing unit 442 stores the reconstructed CT image data and the CT image data generated by various types of image processing in the memory 41.

The reconstruction processing unit 442 calculates energy integration data by determining the sum (total) of the counts of the respective energy bins that are the signals acquired by the detector 12 using the photon-counting method, or the sum of the products of the representative values and counts of the energy bins. The reconstruction processing unit 442 can reconstruct integration image data using the energy integration data. The integration image data is handled as CT image data.

As an application of the medical image capturing apparatus 1 using the photon-counting method, there is a technique for decomposing the types, contents, and densities of materials included in a subject P by using differences in the X-ray absorption characteristics of the respective materials. Such a technique is called material decomposition. For example, the reconstruction processing unit 442 can perform material decomposition on captured data to acquire material decomposition information. The reconstruction processing unit 442 can also reconstruct material decomposition image data indicating decomposed materials by using the material decomposition information that is the result of the material decomposition.

For example, captured data generated from the counting results acquired the medical image capturing apparatus 1 using the photon-counting method includes information about the X-ray energy spectrum attenuated by the transmission through the subject P. The reconstruction processing unit 442 can thus reconstruct material decomposition image data that is an image of specific energy components, for example.

The display control unit 443 displays the integration image data (CT image data) and the material decomposition image data on the display unit 42. The display control unit 443 may select either one of the integration image data (CT image data) and the material decomposition image data and display the selected image data on the display unit 42.

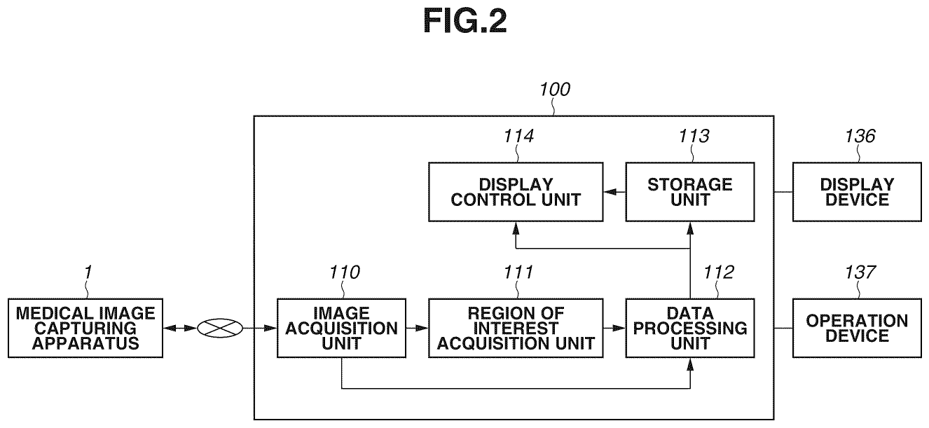

Next, the present exemplary embodiment will be described with reference to FIG. 2. The present exemplary embodiment can be applied to an information processing apparatus 100, a workstation, or an image reading terminal viewer connected to the medical image capturing apparatus 1. Examples of the medical image capturing apparatus 1 include an X-ray CT device using a photon-counting detector and an X-ray CT device using a dual energy detector.

A description will be given of a configuration in which the data amount of image data to be stored in a storage unit 113 of the information processing apparatus 100 is reduced when material decomposition image data (material density image data) obtained by capturing a subject image using the X-ray CT device is stored into the storage unit 113 of the information processing apparatus 100.

The information processing apparatus 100 acquires a plurality of pieces of material decomposition image data generated from detection signals based on the detected numbers of X-ray photons transmitted through a subject P in a plurality of respective energy bands. The information processing apparatus 100 then acquires a region of interest in each of the plurality of pieces of material decomposition image data, and stores partial image data corresponding to the region of interest in each of the plurality of pieces of material decomposition image data in the storage unit 113.

Examples of the materials in the material decomposition image data may include calcium, iodine, and water. However, the materials are not limited thereto and may be any material such as tungsten and titanium. There are four energy bands of 20 keV to 40 keV, 40 keV to 60 keV, 60 keV to 80 keV, and 80 keV to 100 keV, for example, which are set in the medical image capturing apparatus 1 in advance. The number and ranges of energy bands are not limited thereto.

FIG. 2 illustrates a configuration of the information processing apparatus 100 according to the present exemplary embodiment. The information processing apparatus 100 includes an image acquisition unit 110, a region of interest acquisition unit 111, a data processing unit 112, the storage unit 113, and a display control unit 114. The information processing apparatus 100 is connected to the medical image capturing apparatus 1 (X-ray CT device) via a network.

The information processing apparatus 100 includes the image acquisition unit 110 that acquires material decomposition image data from the medical image capturing apparatus 1, and the region of interest acquisition unit 111 that acquires regions of interest in the material decomposition image data. The information processing apparatus 100 also includes the data processing unit 112 that reduces the data amount of the material decomposition image data based on the regions of interest, and the storage unit 113 that stores the material decomposition image data of the reduced data amount.

The image acquisition unit 110 acquires the material decomposition image data from the medical image capturing apparatus 1. The image acquisition unit 110 can acquire data to be used in generation of material decomposition image data in the plurality of energy bands from the medical image capturing apparatus 1. For example, the image acquisition unit 110 can acquire captured data indicating the count value of X-ray photons in each of the plurality of energy bands. The image acquisition unit 110 may also acquire integration image data (CT image data), virtual monochromatic X-ray image data corresponding to virtual single energy imaging, and energy band-specific X-ray image data from the medical image capturing apparatus 1. The image acquisition unit 110 transmits the material decomposition image data, integration image data (CT image data), virtual monochromatic X-ray image data, and/or energy band-specific X-ray image data to the region of interest acquisition unit 111 and the data processing unit 112.

The region of interest acquisition unit 111 uses the material decomposition image data transmitted from the image acquisition unit 110 to acquire regions of interest. As a region of interest, the region of interest acquisition unit 111 acquires a region where a predetermined material is present in the material decomposition image data. With a plurality of pieces of material decomposition image data, the region of interest acquisition unit 111 acquires regions where respective materials are present as regions of interest. For example, the region of interest acquisition unit 111 acquires region of interest masks (1 is set inside the region of interest and 0 is set outside the region of interest) for the regions of interest of the material decomposition image data, and transmits the acquired region of interest masks to the data processing unit 112. Details of the processing will be described below. The region of interest acquisition unit 111 has a function of setting region of interest masks.

The data processing unit 112 acquires partial image data corresponding to the regions of interest of the material decomposition image data transmitted from the image acquisition unit 110, based on the region of interest masks acquired by the region of interest acquisition unit 111. The data processing unit 112 transmits the partial image data to the storage unit 113. The partial image data has a data amount smaller than that of the entire material decomposition image data. The data processing unit 112 may further acquire coordinate information about the partial image data in the material decomposition image data. Details of the processing will be described below.

The storage unit 113 stores the partial image data corresponding to the regions of interest of the material decomposition image data, transmitted from the data processing unit 112. In a case where coordinate information is transmitted from the data processing unit 112, the storage unit 113 stores the coordinate information in association with the partial image data. Storing the coordinate data with the partial image data can reduce the effort for subsequent verification tasks. A display device 136, such as a display, and an operation device 137, such as a mouse and a keyboard, are connected to the information processing apparatus 100. The display control unit 114 can control display of the display device 136.

The information processing apparatus 100 includes a computer including a processor, a memory, and a storage. In such a case, the functions and processing of the image acquisition unit 110, the region of interest acquisition unit 111, the data processing unit 112, and the storage unit 113 are implemented by loading programs stored in the storage into the memory and executing the programs by the processor. However, such a configuration is not restrictive. For example, all or some of the image acquisition unit 110, the region of interest acquisition unit 111, the data processing unit 112, and the storage unit 113 may be implemented by a dedicatedly designed processor (such as an application-specific integrated circuit [ASIC]) or field-programmable gate array (FPGA). Alternatively, part of the arithmetic processing may be performed by a processor, such as a GPU and a digital signal processor (DSP). The information processing apparatus 100 may include a single piece of hardware or a plurality of pieces of hardware. For example, the functions and processing of the information processing apparatus 100 may be implemented by cooperation of a plurality of computers, using cloud computing or distributed computing.

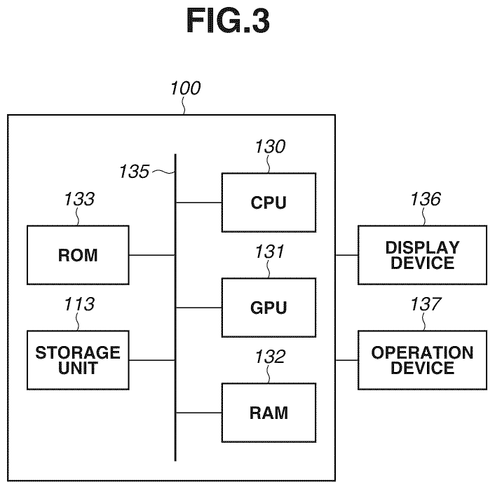

FIG. 3 illustrates an example of the configuration of the information processing apparatus 100. In this example, the information processing apparatus 100 includes a CPU 130, a GPU 131, a RAM 132, a ROM 133, and the storage unit 113, which are connected by a system bus 135. The display device 136, such as a display, and the operation device 137, such as a mouse and a keyboard, are connected to the information processing apparatus 100. The display device 136 has a configuration similar to that of the display unit 42. The operation device 137 has a configuration similar to that of the operation unit 43.

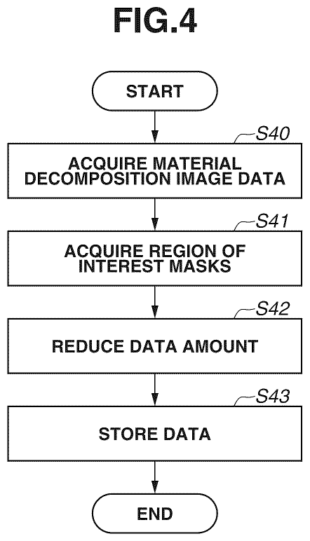

Next, an operation of the information processing apparatus 100 will be described with reference to the flowchart of FIG. 4.

### (Step S40: Data Acquisition)

In step S40, the image acquisition unit 110 acquires material decomposition image data in each of the plurality of energy bands from the medical image capturing apparatus 1. The image acquisition unit 110 transmits the material decomposition image data to the region of interest acquisition unit 111 and the data processing unit 112. The image acquisition unit 110 may also acquire integration image data (CT image data) from the medical image capturing apparatus 1.

### (Step S41: Acquisition of Regions of Interest)

In step S41, the region of interest acquisition unit 111 sets and acquires region of interest masks for the regions of interest in the respective pieces of material decomposition image data, based on the plurality of pieces of material decomposition image data. The region of interest acquisition unit 111 transmits the region of interest masks corresponding to respective materials to the data processing unit 112.

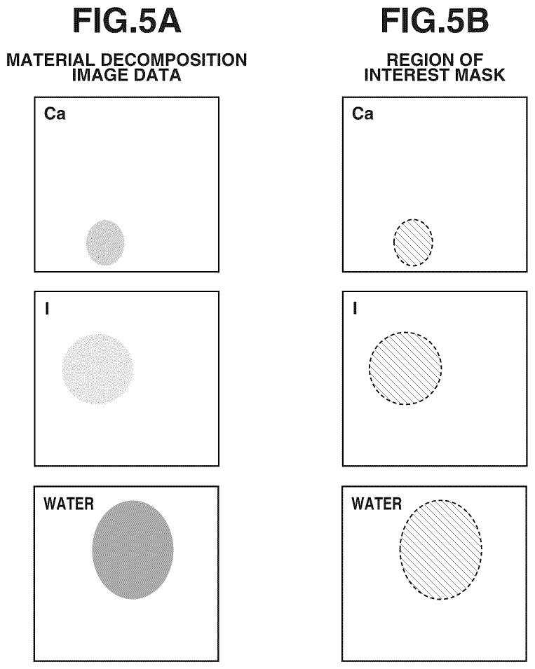

The region of interest acquisition unit 111 will be described with reference to FIGS. 5A and 5B. FIGS. 5A and 5B are diagrams illustrating the acquisition of the region of interest masks corresponding to the regions of interest of the material decomposition image data.

FIG. 5A illustrates a plurality of pieces of material decomposition image data in a plurality of respective energy bands. While the description will be given of material decomposition image data on calcium, iodine, and water, material decomposition image data on materials other than calcium, iodine, or water may be used.

The region of interest acquisition unit 111 acquires a region of interest in each piece of material decomposition image data, and sets a region of interest mask. The region of interest acquisition unit 111 sets a threshold about a predetermined physical quantity for each piece of material decomposition image data, and acquires a region where the physical quantity is higher than or equal to the threshold, as a region of interest. For example, the region of interest acquisition unit 111 sets a threshold about a material density or pixel value for each piece of material decomposition image data, and acquires a region where the material density or pixel value is higher than or equal to the threshold, as a region of interest. For example, the region of interest acquisition unit 111 sets a value one half of the maximum possible material density in each piece of material decomposition image data as the threshold, and acquires a region where the material density is higher than or equal to the threshold, as a region of interest. The region of interest acquisition unit 111 then sets a region of interest mask for the region where the material density is higher than or equal to the threshold in each piece of material decomposition image data.

For example, the region of interest acquisition unit 111 sets a threshold about the material density for material decomposition image data on calcium, and acquires a region where the material density is higher than or equal to the threshold, as a region of interest. The region of interest acquisition unit 111 then sets a region of interest mask for the region where the material density is higher than or equal to the threshold in the material decomposition image data on calcium. Further, for example, the region of interest acquisition unit 111 sets a threshold about the material density for the material decomposition image data on iodine, and acquires a region where the material density is higher than or equal to the threshold, as a region of interest.

The region of interest acquisition unit 111 then sets a region of interest mask for the region where the material density is higher than or equal to the threshold in the material decomposition image data on iodine. A region of interest mask for water is set in a manner similar to the foregoing.

Alternatively, for example, the region of interest acquisition unit 111 can set a predetermined pixel value in each piece of material decomposition image data as a threshold, and acquire a region where the pixel value is higher than or equal to the predetermined pixel value, as a region of interest. The region of interest acquisition unit 111 then sets a region of interest mask for each piece of material decomposition image data.

For example, the region of interest acquisition unit 111 sets a threshold corresponding to a predetermined pixel value in the material decomposition image data on calcium, and acquires a region having pixels values higher than or equal to the threshold as a region of interest. The region of interest acquisition unit 111 sets a region of interest mask for the region having pixel values higher than or equal to the threshold in the material decomposition image data on calcium. Further, for example, the region of interest acquisition unit 111 sets a threshold corresponding to a predetermined pixel value in the material decomposition image data on iodine, and acquires a region having pixel values higher than or equal to the threshold, as a region of interest. The region of interest acquisition unit 111 sets a region of interest mask for the region having pixel values higher than or equal to the threshold in the material decomposition image data on iodine. A region of interest mask for water is set in a manner similar to the foregoing.

FIG. 5B illustrates the region of interest masks for the respective pieces of material decomposition image data.

The region of interest masks are indicated by hatching. In the present exemplary embodiment, 1 is set inside the regions of interest, and 0 is set outside the regions of interest.

For the material decomposition image data on calcium, a region where calcium is extracted at or above a predetermined level is set as the region of interest mask. For the material decomposition image data on iodine, a region where iodine is extracted at or above a predetermined level is set as the region of interest mask. For the material decomposition image data on water, a region where water is extracted at or above a predetermined level is set as the region of interest mask.

Aside from the foregoing threshold-based technique, the region of interest masks may be set using any technique for extracting a region of interest from material decomposition image data. For example, a deep learning-based region extraction technique may be used.

In the deep learning-based region extraction technique, the region of interest acquisition unit 111 uses a neural network (trained model) for a segmentation task, for example. The region of interest acquisition unit 111 acquires training data where material decomposition image data is paired with correct answer labels indicating target materials in the material decomposition image data. Specifically, the region of interest acquisition unit 111 acquires training data where position information about pixels of target materials in the material decomposition image data is paired with correct answer images to which correct answer labels serving as labels indicating the target materials are attached. The region of interest acquisition unit 111 makes inference on each piece of material decomposition image data using the trained model, and acquires a region of interest.

The region of interest acquisition unit 111 acquires a region inferred (segmented) to be calcium in the material decomposition image data on calcium as a region of interest using the trained model, and sets a region of interest mask. The region of interest acquisition unit 111 acquires a region inferred (segmented) to be iodine in the material decomposition image data on iodine as a region of interest using the trained model, and sets a region of interest mask. The region of interest acquisition unit 111 acquires a region inferred (segmented) to be water in the material decomposition image data on water as a region of interest using the trained model, and sets a region of interest mask.

The region of interest masks may take forms other than images. For example, a region of interest mask may be expressed by coordinates of a group of dots representing the boundary of the region of interest. The information expressing a region of interest may take forms other than a region of interest mask. For example, the information may be image data (likelihood image data) where the likelihood of a region of interest is set in a pixel basis (for example, in values of 0 to 1). Likelihood values of 0 to 1 may be set inside a region of interest, and a value of 0 may be set outside the region of interest. Furthermore, a region of interest does not need to express the exact shape of the actual object of interest and may be shaped to express a rough shape of the actual object. For example, a region of interest may have a shape surrounding the actual object or a shape such as an elliptic or rectangular shape.

The region of interest acquisition unit 111 may acquire the regions of interest in the respective pieces of material decomposition image data and not set a region of interest mask.

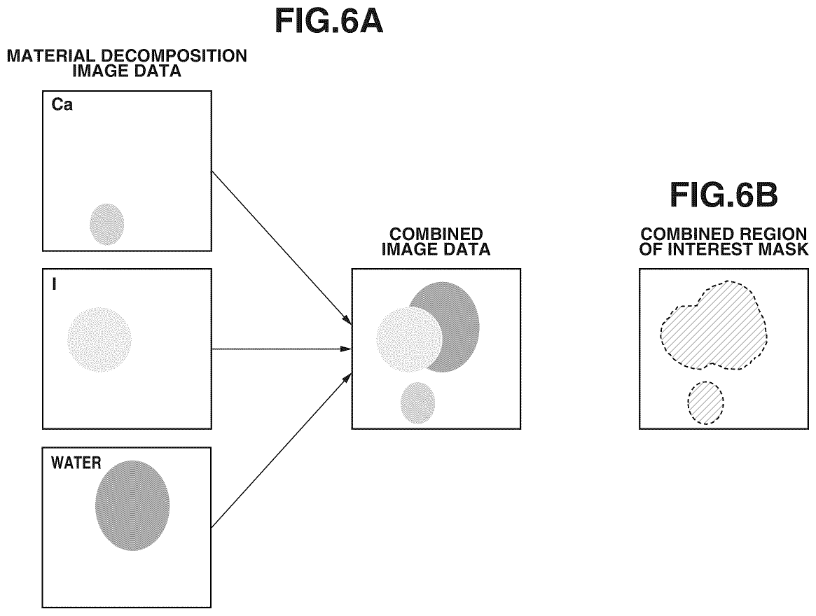

FIGS. 6A and 6B are diagrams illustrating the acquisition of a combined region of interest mask for combined image data obtained by combining a plurality of pieces of material decomposition image data. FIG. 6A illustrates a plurality of pieces of material decomposition image data in a plurality of respective energy bands, and combined image data obtained by combining the plurality of pieces of material decomposition image data.

The region of interest acquisition unit 111 generates the combined image data from the plurality of pieces of material decomposition image data by using a conventional method. The region of interest acquisition unit 111 generates the combined image data by aligning the plurality of pieces of material decomposition image data with each other and combining the plurality of pieces of material decomposition image data. The region of interest acquisition unit 111 acquires a combined region of interest mask by performing threshold processing on the combined image data.

The region of interest acquisition unit 111 acquires a region of interest from the combined image data obtained by combining the plurality of pieces of material decomposition image data, and sets the combined region of interest mask. The region of interest acquisition unit 111 sets a threshold about a predetermined physical quantity (material density or pixel value) for the combined image data, and acquires a region where the physical quantity is higher than or equal to the threshold, as the combined region of interest. For example, the region of interest acquisition unit 111 sets a value one half of the maximum possible material density in the combined image data as a threshold, and acquires a region where the material density is higher than or equal to the threshold, as the combined region of interest. The region of interest acquisition unit 111 then sets a combined region of interest mask for the combined image data. Alternatively, the region of interest acquisition unit 111 can set a threshold corresponding to a predetermined pixel value for the combined image data, and acquire a region where the pixel value is higher than or equal to the threshold, as the combined region of interest.

The data processing unit 112 generates partial image data of the combined image data based on the combined region of interest mask. The storage unit 113 stores the partial image data corresponding to the combined region of interest in the combined image data.

FIG. 6B illustrates the combined region of interest mask for the combined image data obtained by combining the plurality of pieces of material decomposition image data. The combined region of interest mask is indicated by hatching. The combined region of interest mask is configured so that 1 is set inside the combined region of interest, and 0 is set outside the combined region of interest.

The region of interest acquisition unit 111 may set the combined region of interest mask by acquiring region of interest masks from the plurality of pieces of material decomposition image data and combining the region of interest masks.

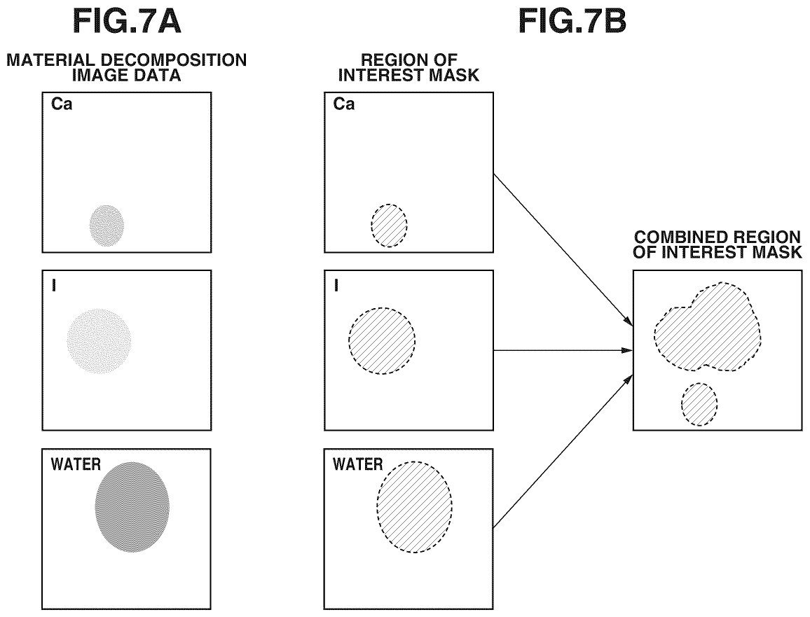

The acquisition of the combined region of interest mask will be described with reference to FIGS. 7A and 7B. FIG. 7A illustrates a plurality of pieces of material decomposition image data in a plurality of respective energy bands. FIG. 7B illustrates region of interest masks for the respective pieces of material decomposition image data and a combined region of interest mask obtained by combining the plurality of region of interest masks.

As described with reference to FIGS. 5A and 5B, the region of interest acquisition unit 111 acquires a region of interest in each of the pieces of material decomposition image data. The region of interest acquisition unit 111 sets a threshold about a predetermined physical quantity (material density or pixel value) for each piece of material decomposition image data, and acquires a region where the physical quantity is higher than or equal to the threshold, as a region of interest. The region of interest acquisition unit 111 then sets a region of interest mask for each piece of material decomposition image data.

The region of interest acquisition unit 111 aligns the region of interest masks for the respective pieces of material decomposition image data with each other and combines the region of interest masks. The region of interest acquisition unit 111 sets a combined region of interest mask into which the region of interest masks are combined. The combined region of interest mask is configured so that 1 is set inside the combined region of interest and 0 is set outside the combined region of interest.

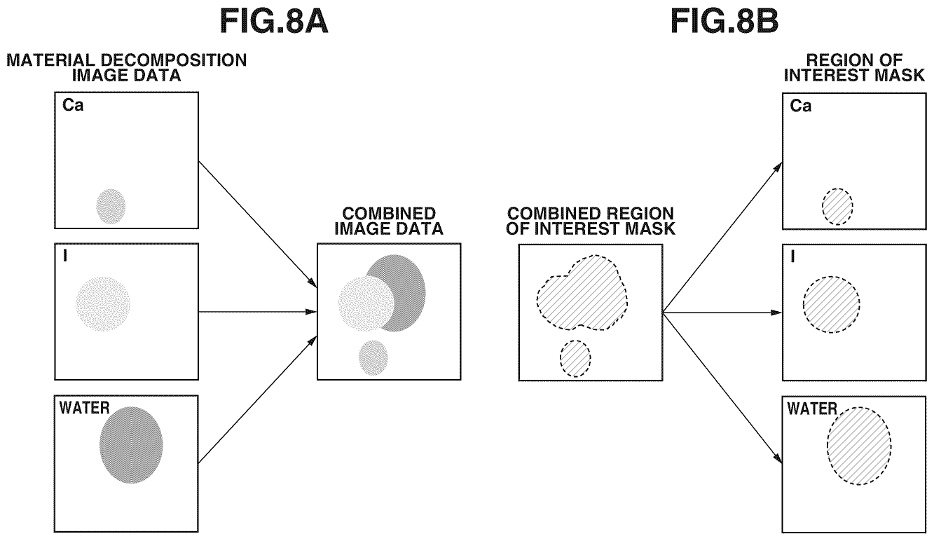

FIGS. 8A and 8B are diagrams illustrating the acquisition of a combined region of interest in combined image data obtained by combining a plurality of pieces of material decomposition image data and the acquisition of regions of interest in the plurality of pieces of material decomposition image data. FIG. 8A illustrates a plurality of pieces of material decomposition image data in a plurality of respective energy bands and combined image data obtained by combining the plurality of pieces of material decomposition image data. FIG. 8B illustrates a combined region of interest mask and region of interest masks for respective materials. The process up to the setting of the combined region of interest mask is similar to that of FIGS. 6A and 6B. A description thereof will thus be omitted.

The region of interest acquisition unit 111 sets region of interest masks of the respective materials based on position information about the combined region of interest mask. For example, the region of interest acquisition unit 111 uses position information about the presence of calcium and the position information about the combined region of interest mask to acquire a region of interest of calcium in the material decomposition image data on calcium. As the region of interest of calcium, the region of interest acquisition unit 111 acquires a region where the position at which calcium is present overlaps with the position of the combined region of interest mask. The region of interest acquisition unit 111 sets a region of interest mask for the region of interest of calcium.

For example, the region of interest acquisition unit 111 uses position information about the presence of iodine and the position information about the combined region of interest mask to acquire a region of interest of iodine in the material decomposition image data on iodine.

As the region of interest of iodine, the region of interest acquisition unit 111 acquires a region where the position at which iodine is present overlaps with the position of the combined region of interest mask. The region of interest acquisition unit 111 sets a region of interest mask for the region of interest of iodine. A region of interest mask for water is set in a manner similar to the foregoing.

FIG. 8B illustrates the region of interest masks for the respective pieces of material decomposition image data.

The region of interest masks are configured so that 1 is set inside the region of interest and 0 is set outside the region of interest.

The region of interest acquisition unit 111 may perform smoothing operation on the region of interest masks, perform opening operation or closing operation, and/or apply other image processing.

The smoothing operation is to reduce differences between adjoining pixel values in image data representing a region of interest mask so that the image data includes a smoothly continuous set of data. This can reduce singularities and noise. The opening operation involves erosion of the image data representing a region of interest mask a plurality times and dilation of the image data. This processing can reduce noise. The closing operation involves dilation on the image data representing a region of interest mask a plurality of times and erosion of the image data. This processing can reduce noise.

### (Step S42: Reduction of Data Amount)

In step S42, the data processing unit 112 reflects the region of interest masks acquired by the region of interest acquisition unit 111 on the material decomposition image data to reduce the data amount. The data processing unit 112 acquires the partial image data in the regions of interest of the material decomposition image data, and deletes the image data in the regions other than the regions of interest of the material decomposition image data. Since the regions other than the regions of interest of the material decomposition image data are deleted, the data amount is reduced.

For example, the data processing unit 112 sets storage regions including the regions of interest represented by the region of interest masks, and generates partial image data by extracting the storage regions. The data processing unit 112 transmits the partial image data (including coordinate information about the storage regions) of the material decomposition image data to the storage unit 113. The storage regions may be regions circumscribing the regions of interest, or ones formed by attaching a predetermined margin to around the regions of interest. In a case where the regions of interest acquired in step S41 are likelihood images, the data processing unit 112 can set storage regions including the regions where the likelihood exceeds a predetermined value, and generate partial image data by extracting the storage regions.

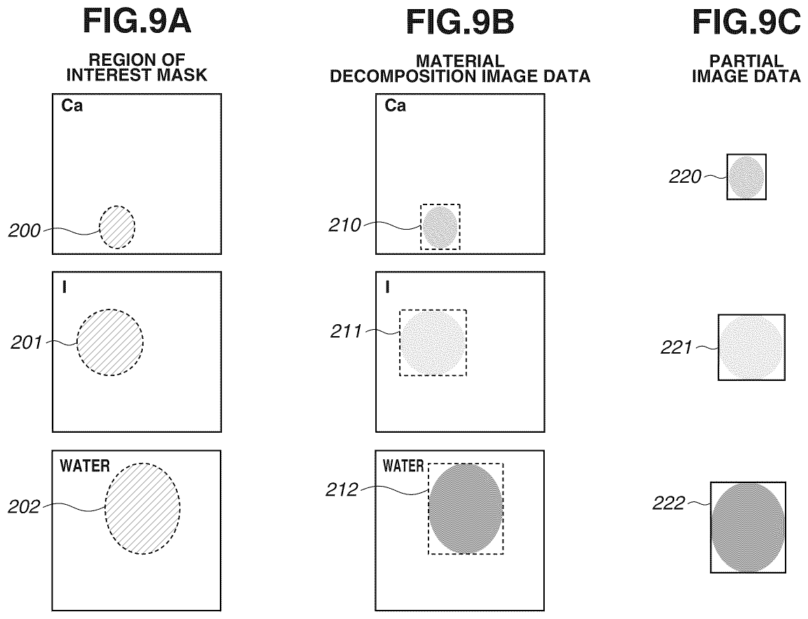

FIGS. 9A to 9C are diagrams illustrating an example of data reduction processing by the data processing unit 112. FIG. 9A is a diagram illustrating region of interest masks. FIG. 9B is a diagram illustrating storage regions in material decomposition image data. FIG. 9C is a diagram illustrating partial image data to be stored in the storage unit 113. The data processing unit 112 may further acquire coordinate information about the partial image data.

As illustrated in FIG. 9A, the region of interest acquisition unit 111 acquires a region of interest in the material decomposition image data on calcium, and sets a region of interest mask 200 for the material decomposition image data on calcium. The region of interest acquisition unit 111 acquires a region of interest in the material decomposition image data on iodine, and sets a region of interest mask 201 for the material decomposition image data on iodine. Similarly, the region of interest acquisition unit 111 acquires a region of interest in the material decomposition image data on water, and sets a region of interest mask 202 for the material decomposition image data on water.

As illustrated in FIG. 9B, the data processing unit 112 sets storage regions including the regions of interest represented by the region of interest masks, and generates partial image data by extracting the storage regions. The data processing unit 112 sets a storage region 210 including the region of interest mask 200 set for the material decomposition image data on calcium. In the present exemplary embodiment, the data processing unit 112 sets the storage region 210 including the region of interest mask 200 in a rectangular shape. The data processing unit 112 sets a storage region 211 including the region of interest mask 201 set for the material decomposition image data on iodine. In the present exemplary embodiment, the data processing unit 112 sets the storage region 211 including the region of interest mask 201 in a rectangular shape. Similarly, the data processing unit 112 sets a storage region 212 including the region of interest mask 202 set for the material decomposition image data on water. In the present exemplary embodiment, the data processing unit 112 sets the storage region 212 including the region of interest mask 202 in a rectangular shape. Each storage region may include coordinate information.

As illustrated in FIG. 9C, the data processing unit 112 generates partial image data 220 by extracting the storage region 210 from the material decomposition image data on calcium. The data processing unit 112 transmits the partial image data 220 of the material decomposition image data on calcium (including coordinate information in the material decomposition image data) to the storage unit 113. The data processing unit 112 generates partial image data 221 by extracting the storage region 211 from the material decomposition image data on iodine. The data processing unit 112 transmits the partial image data 221 of the material decomposition image data on iodine (including coordinate information in the material decomposition image data) to the storage unit 113. Similarly, the data processing unit 112 generates partial image data 222 by extracting the storage region 212 from the material decomposition image data on water. The data processing unit 112 transmits the partial image data 222 of the material decomposition image data on water (including coordinate information in the material decomposition image data) to the storage unit 113.

In the present exemplary embodiment, the regions of interest identified for the plurality of respective pieces of material decomposition image data are stored. The regions of interest of the material decomposition image data may differ from one piece of material decomposition image data to another.

In a case where there is a plurality of regions of interest in a piece of material decomposition image data, a storage region including (in combination of) all the regions of interest may be set. Storage regions including the respective regions of interest may be set on a one-on-one basis. Adjoining regions of interest may be grouped, and a storage region may be set for each group.

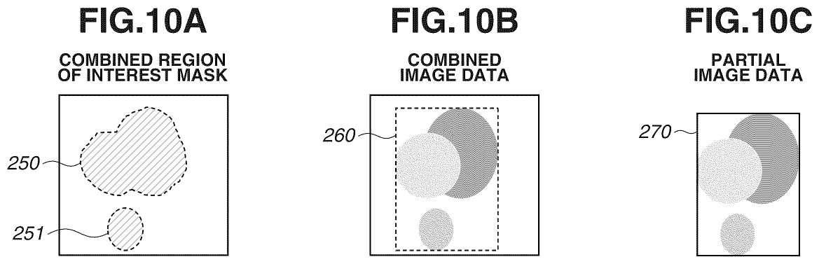

FIGS. 10A to 10C are diagrams illustrating an example of the data reduction processing by the data processing unit 112. FIG. 10A is a diagram illustrating a plurality of region of interest masks 250 and 251 for combined image data of material decomposition image data. FIG. 10B is a diagram illustrating a storage region 260 in the combined image data of the material decomposition image data. FIG. 10C is a diagram illustrating partial image data 270 to be stored in the storage unit 113.

As illustrated in FIG. 10A, the region of interest acquisition unit 111 acquires regions of interest in combined image data of a plurality of pieces of material decomposition image data, and sets the plurality of region of interest masks 250 and 251 for the combined image data.

As illustrated in FIG. 10B, the data processing unit 112 sets the storage region 260 including the regions of interest represented by the plurality of region of interest masks 250 and 251. The data processing unit 112 then generates partial image data 270 by extracting the storage region 260 from the combined image data of the plurality of pieces of material decomposition image data. In the present exemplary embodiment, the data processing unit 112 sets the storage region 260 including the plurality of region of interest masks 250 and 251 in a rectangular shape.

As illustrated in FIG. 10C, the data processing unit 112 generates the partial image data 270 by extracting the storage region 260 from the combined image data of the plurality of pieces of material decomposition image data. The data processing unit 112 transmits the partial image data 270 of the combined image data (including coordinate information in the material decomposition image data) to the storage unit 113.

The storage region is not limited to the rectangular shape, and may have a predetermined shape such as a circle or an ellipse to include the region(s) of interest, or any given shape. In any case, the partial image data can be expressed by information expressing the shape of the region and vector data expressing the pixel values of the respective pixels within the shape in the form of a one-dimensional array.

In a case where the storage region has a predetermined shape, parameters expressing the shape, such as a center position and a radius, apply to the information expressing the shape of the region. In a case where the storage region has an indefinite shape, the information about the mask representing the shape applies to the information expressing the shape of the region. In the latter case, the region of interest mask(s) itself/themselves can be used as the information expressing the shape of the storage region. In other words, the data processing unit 112 can simply use the region(s) of interest as the storage region.

### (Step S43: Data Storage)

In step S43, the storage unit 113 stores (saves) the partial image data of the material decomposition image data acquired from the data processing unit 112.

The storage unit 113 stores the partial image data of the material decomposition image data in a material basis. As illustrated in FIGS. 9A to 9C, the storage unit 113 stores the partial image data 220 of the material decomposition image data on calcium. The storage unit 113 stores the partial image data 221 of the material decomposition image data on iodine. The storage unit 113 stores the partial image data 222 of the material decomposition image data on water.

As illustrated in FIGS. 10A to 10C, the storage unit 113 can store the partial image data 270 obtained by extracting the storage region 260 from the combined image data of the plurality of pieces of material decomposition image data.

The information processing apparatus 100 can read the partial image data of the material decomposition image data from the storage unit 113, and display the partial image data of the material decomposition image data on the display device 136 via the display control unit 114. In a case where the user issues an instruction to display material decomposition image data on a material to be identified via the operation device 137, the partial image data of the material decomposition image data on the material to be identified is read from the storage unit 113. The display control unit 114 can display the partial image data of the material decomposition image data on the material to be identified on the display device 136.

For example, in a case where the user issues an instruction to display the material decomposition image data on calcium via the operation device 137, the partial image data of the material decomposition image data on calcium is read from the storage unit 113. The display control unit 114 can display the partial image data of the material decomposition image data on calcium on the display device 136. In a case where the user issues an instruction to display the material decomposition image data on iodine via the operation device 137, the partial image data of the material decomposition image data on iodine is read from the storage unit 113. The display control unit 114 can display the partial image data of the material decomposition image data on iodine on the display device 136. The partial image data of the material decomposition image data on water is displayed in a manner similar to the foregoing.

The information processing apparatus 100 according to the present exemplary embodiment includes the image acquisition unit 110 that acquires material decomposition image data based on the detection signals related to X-ray photons transmitted through the subject P in a plurality of respective energy bands. The information processing apparatus 100 also includes the region of interest acquisition unit 111 that acquires regions of interest in the material decomposition image data. The information processing apparatus 100 further includes the data processing unit 112 that generates partial image data of the material decomposition image data corresponding to the regions of interest, and the storage unit 113 that stores the partial image data of the material decomposition image data. Such a configuration can reduce the memory consumption data amount of the material decomposition image data.

### (First Modification of First Exemplary Embodiment)

In the above-described exemplary embodiment, the storage unit 113 stores the region of interest of each of the plurality of pieces of material decomposition image data in the processing of step S43. However, the present exemplary embodiment is not limited thereto. The information processing apparatus 100 may include a selection unit (operation device 137) for selecting a material to be identified, and the storage unit 113 may be configured to store the region(s) of interest of at least one or more pieces of material decomposition image data selected by the user. In other words, the storage unit 113 may be configured to not store the region(s) of interest of the material decomposition image data on a material or materials not selected by the user.

As a method for selecting at least one piece of material decomposition image data, the selection unit may select material decomposition image data on a material or materials selected by the user in advance. Alternatively, the plurality of pieces of material decomposition image data may be analyzed, and the selection unit may select a piece of material decomposition image data that is drawn with the highest density. The storage unit 113 may be configured to store the regions of interest of a plurality of pieces of material decomposition image data that satisfies a predetermined condition (for example, that the density be higher than or equal to a predetermined value).

For example, in a case where iodine is selected by the user using the selection unit as the material to be identified, as illustrated in FIGS. 9A to 9C, the storage unit 113 stores the partial image data 221 corresponding to the region of interest of the material decomposition image data on iodine. In a case where the X-ray CT device is used to check the effect of an iodinated contrast agent (iodine compound), the storage unit 113 stores the partial image data 221 corresponding to the region of interest of the material decomposition image data on iodine, and deletes the rest of the material decomposition image data. Alternatively, the storage unit 113 may store the partial image data 221 corresponding to the region of interest of the material decomposition image data on iodine and integration image data to be described below, and delete the material decomposition image data on other than iodine, i.e., the material decomposition image data on calcium and water.

The storage unit 113 may be configured to store the entire material decomposition image data on the material to be identified selected by the user using the selection unit, instead of the partial image data. In such a case, the processing of step S41 where the region of interest acquisition unit 111 acquires regions of interest may be omitted. For example, in a case where iodine is selected by the user using the selection unit as the material to be identified, the storage unit 113 stores the material decomposition image data on iodine. In a case where calcium is selected by the user using the selection unit as the material to be identified, the storage unit 113 stores the material decomposition image data on calcium. Since the material decomposition image data on the material to be identified selected by the user using the selection unit is stored in the storage unit 113, the memory consumption data amount of the material decomposition image data can be reduced.

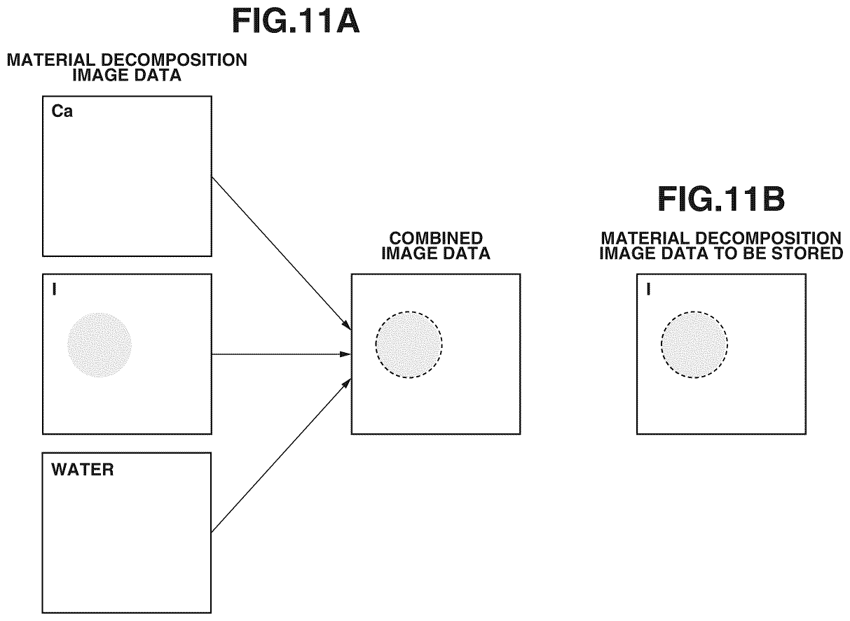

FIGS. 11A and 11B are diagrams illustrating a mode where the material decomposition image data on the material to be identified selected by the user using the selection unit is stored in the storage unit 113. Based on the material to be identified selected by the user using the selection unit, the storage unit 113 stores the material decomposition image data on the material to be identified and does not store the material decomposition image data on materials other than that to be identified.

As illustrated in FIGS. 11A and 11B, in a case where iodine is selected by the user using the selection unit as the material to be identified, the storage unit 113 stores the material decomposition image data on iodine. The material decomposition image data on other than iodine, namely, the material decomposition image data on calcium and water is not stored in the storage unit 113. Since the storage unit 113 stores the material decomposition image data on iodine and does not store the material decomposition image data on other than iodine, the memory consumption data amount of the material decomposition image data can be reduced.

In a case where calcium and iodine are selected by the user using the selection unit as the material to be identified, the storage unit 113 stores the material decomposition image data on calcium and the material decomposition image data on iodine. The material decomposition image data on other than calcium or iodine, i.e., the material decomposition image data on water is not stored in the storage unit 113. Since the storage unit 113 stores the material decomposition image data on calcium and iodine and does not store the material decomposition image data on other than calcium or iodine, the memory consumption data amount of the material decomposition image data can be reduced.

The storage unit 113 may be configured to, even when a material to be identified is selected by the user using the selection unit, not store the material decomposition image data on that material in a case where the region of the material to be identified related to the material decomposition image data on the material to be identified is less than a predetermined range (area). In a case where the region of the material to be identified related to the material decomposition image data on the material to be identified is greater than or equal to the predetermined range (area), the storage unit 113 stores the material decomposition image data on that material.

Since the storage unit 113 does not store noisy material decomposition image data on the material to be identified, the memory consumption data amount of the material decomposition image data can be reduced.

As described above, the present exemplary embodiment includes the image acquisition unit 110 that acquires a plurality of pieces of material decomposition image data based on the detection signals related to X-ray photons transmitted through the subject P in a plurality of respective energy bands, and the selection unit (operation device 137) for selecting a material to be identified. The present exemplary embodiment also includes the storage unit 113 that stores at least one piece of material decomposition image data from among the plurality of pieces of material decomposition image data, based on the selected material to be identified.

### (Second Modification of First Exemplary Embodiment)

In the above-described exemplary embodiment, in step S43, the storage unit 113 stores the partial image data corresponding to the regions of interest of the material decomposition image data. However, the present exemplary embodiment is not limited thereto. The storage unit 113 may store spatial information, such as spatial positions of respective voxels in a region of interest of the material decomposition image data, and luminance values in association with each other. This enables display of the region of interest at an appropriate position due to the presence of the spatial information and the luminance values of the voxels of the region of interest even in a case where there is no voxel outside the region of interest of the material decomposition image data.

### (Third Modification of First Exemplary Embodiment)

In the above-described exemplary embodiment, in step S40, the image acquisition unit 110 acquires the material decomposition image data from the detection signals. However, the present exemplary embodiment is not limited thereto. For example, the image acquisition unit 110 may acquire virtual monochromatic X-ray image data corresponding to virtual monochromatic energy imaging or integration image data obtained by reconstructing energy band-specific X-ray image data from the detection signals. Alternatively, the image acquisition unit 110 may acquire integration image data by reconstructing energy integration signals that are obtained by adding the detection signals indicating the numbers of X-ray photons detected in the plurality of respective energy bands in a detector basis.

The regions of interest that can be identified may differ between cases of when the images obtained by the image acquisition unit 110 are material decomposition image data, virtual monochromatic X-ray image data, energy band-specific X-ray image data, or integration image data. Even with image data other than the material decomposition image data, the memory consumption data amount can be similarly reduced by identifying the regions of interest.

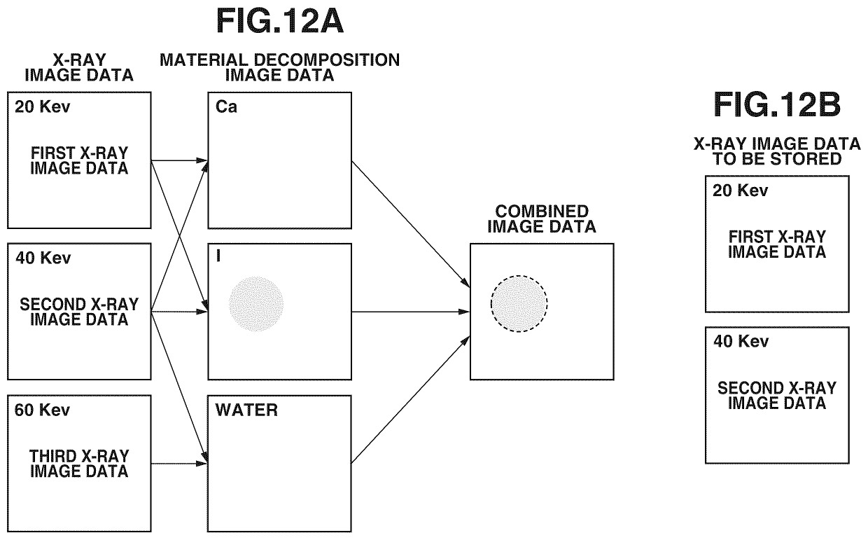

FIGS. 12A and 12B are diagrams illustrating a mode where energy band-specific X-ray image data is stored. FIG. 12A illustrates a correspondence between material decomposition image data and X-ray image data. FIG. 12B illustrates X-ray image data to be stored.

Material decomposition image data includes energy band-specific X-ray image data. In a case the material selected by the user using the selection unit (operation device 137) is iodine, the storage unit 113 stores energy band-specific X-ray image data that relates to (for example, can best express) the material decomposition image data on iodine.

Energy band-specific X-ray image data includes a plurality of pieces of the X-ray image data. For example, first X-ray image data is low-energy captured image data corresponding to a low tube voltage. Second X-ray image data is high-energy captured image data corresponding to a high tube voltage. For example, the information processing apparatus 100 generates first material decomposition image data indicating the distribution of a first material by material decomposition processing, based on the first X-ray image data and the second X-ray image data. The information processing apparatus 100 generates second material decomposition image data indicating the distribution of a second material by the material decomposition processing, based on the second X-ray image data and third X-ray image data.

In the present exemplary embodiment, the material decomposition image data indicating the distribution of calcium is generated by the material decomposition processing, based on the first X-ray image data and the second X-ray image data. Also, the material decomposition image data indicating the distribution of iodine is generated by the material decomposition processing, based on the first X-ray image data and the second X-ray image data. Also, the material decomposition image data indicating the distribution of water is generated by the material decomposition processing, based on the second X-ray image data and the third X-ray image data.

As illustrated in FIGS. 12A and 12B, in a case where the material selected by the user using the selection unit is iodine, the storage unit 113 stores the first X-ray image data and the second X-ray image data related to the material decomposition image data on iodine.

In a case where the material selected by the user using the selection unit is calcium, the storage unit 113 stores the first X-ray image data and the second X-ray image data related to the material decomposition image data on calcium. In a case where the material selected by the user is water, the storage unit 113 stores the second X-ray image data and the third X-ray image data related to the material decomposition image data on water.

As described above, the present exemplary embodiment includes the image acquisition unit 110 that acquires a plurality of pieces of material decomposition image data generated from X-ray image data in a plurality of energy bands based on the detection signals related to X-ray photons transmitted through the subject P in the plurality of respective energy bands. The present exemplary embodiment also includes the selection unit (operation device 137) for selecting the material to be identified. The present exemplary embodiment further includes the storage unit 113 that stores the energy band-specific X-ray image data from which the material decomposition image data related to the selected material to be identified is generated.

Next, a second exemplary embodiment will be described. A difference from the first exemplary embodiment is that the storage unit 113 stores integration image data as well as partial image data corresponding to the regions of interest in the material decomposition image data.

In the present exemplary embodiment, material decomposition image data based on detection signals obtained by a medical image capturing apparatus 1 capturing the chest and abdomen of a subject is combined with integration image data obtained by converting the detection signals into energy integration signals and reconstructing the energy integration signals, and the combined image data is displayed.

The materials on which material decomposition image data is generated, and the energy bands are similar to those in the first exemplary embodiment.

Specifically, the materials on which material decomposition image data is generated are the following four: calcium, iodine, water, and air. The energy bands are the following five: 20 keV to 40 keV, 40 keV to 60 keV, 60 keV to 80 keV, 80 keV to 100 keV, and 100 keV to 120 keV.

Details of the components of an information processing apparatus 100 according to the present exemplary embodiment will now be described with reference to FIG. 2. In the present exemplary embodiment, the storage unit 113 stores material decomposition image data based on region of interest masks, and stores integration image data irrespective of the region of interest masks. The material decomposition image data inside the regions of interest is generated as partial image data. The storage unit 113 stores the partial image data of the material decomposition image data, and stores the integration image data inside and outside the regions of interest (entire image). In other words, a data processing unit 112 performs data amount reduction processing (data reduction processing) by deleting the material decomposition image data outside the regions of interest. The data amount reduction processing is not performed on the integration image data.

A display control unit 114 generates combined image data by combining the partial image data of the material decomposition image data with the integration image data, and displays the combined image data on a display device 136.

### (Image Acquisition Unit 110)

An image acquisition unit 110 acquires material decomposition image data in each of a plurality of energy bands and integration image data from a medical image capturing apparatus 1. The image acquisition unit 110 transmits the material decomposition image data and the integration image data to a region of interest acquisition unit 111 and the data processing unit 112.

### (Region of Interest Acquisition Unit 111)

The region of interest acquisition unit 111 sets a threshold about a predetermined physical quantity for each of the plurality of pieces of material decomposition image data, and acquires a single region of interest mask as a single region of interest by setting a region where the physical quantity is higher than or equal to the threshold. The region of interest acquisition unit 111 then acquires a combined region of interest mask by combining the single region of interest masks, and transmits the combined region of interest mask to the data processing unit 112.

### (Data Processing Unit 112)

The data processing unit 112 acquires partial image data of each piece of material decomposition image data corresponding to a combined region of interest using the combined region of interest mask acquired from the region of interest acquisition unit 111, and transmits the partial image data to the storage unit 113 and the display control unit 114. In the present exemplary embodiment, the integration image data is transmitted to the storage unit 113 and the display control unit 114 without the data reduction processing.

### (Storage Unit 113)

The storage unit 113 stores the partial image data of each piece of material decomposition image data and the integration image data.

### (Display Control Unit 114)

The display control unit 114 generates combined image data by combining the partial image data of each piece of material decomposition image data and the integration image data acquired from the data processing unit 112 at a certain cross section, and displays the combined image data on the display device 136. Details of the processing will be described below in conjunction with the description of step S54.

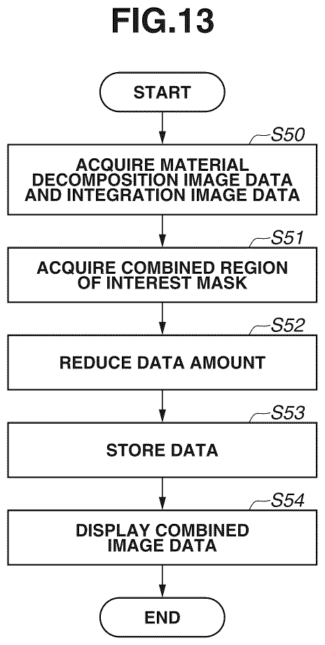

Next, an operation of the information processing apparatus 100 will be described with reference to the flowchart of FIG. 13.

### (Step S50: Data Acquisition)

In step S50, the image acquisition unit 110 acquires the material decomposition image data in the plurality of respective energy bands and the integration image data from the medical image capturing apparatus 1, and transmits the material decomposition image data and the integration image data to the region of interest acquisition unit 111 and the data processing unit 112.

Instead of acquiring the material decomposition image data and the integration image data, the image acquisition unit 110 may acquire data by the following method. Initially, the image acquisition unit 110 acquires the detection signals based on the numbers of X-ray photons that have been transmitted through the subject and detected in the plurality of respective energy bands, from the medical image capturing apparatus 1. The image acquisition unit 110 acquires energy integration signals obtained by adding the detection signals in a detector basis and reconstructs the material decomposition image data and the integration image data.

In the present exemplary embodiment, the material decomposition image data and the integration image data can be reconstructed (generated) by back-projecting the detection signals by using an image reconstruction algorithm. Any image reconstruction algorithm may be used here. Examples include analytical image reconstruction methods based on the FBP and universal back projection (UBP), and statistical image reconstruction methods based on maximum likelihood expectation maximization (ML-EM) and ordered subset expectation maximization (OS-EM).

### (Step S51: Acquisition of Combined Region of Interest)

In step S51, similar to the first exemplary embodiment, the region of interest acquisition unit 111 sets region of interest masks for the respective pieces of material decomposition image data. The region of interest acquisition unit 111 acquires a combined region of interest mask by combining the plurality of region of interest masks, and transmits the combined region of interest mask to the data processing unit 112. As a method for integrating the masks, for example, logical OR, majority voting, or logical AND can be used.

### (Step S52: Reduction of Data Amount)

In step S52, like the first exemplary embodiment, the data processing unit 112 acquires the partial image data in the regions of interest of the material decomposition image data, and transmits the partial image data to the storage unit 113 and the display control unit 114. In the present exemplary embodiment, the combined region of interest mask is used instead of the region of interest masks for the respective pieces of material decomposition image data. In this step, the data processing unit 112 transmits the integration image data to the storage unit 113 and the display control unit 114 without performing the data amount reduction processing.

### (Step S53: Data Storage)

In step S53, the storage unit 113 stores the data of the reduced data amount (i.e., the partial image data of each piece of material decomposition image data and the integration image data).

### (Step S54: Data Display)

In step S54, the display control unit 114 generates combined image data by combining the partial image data of each piece of material decomposition image data with the integration image data, and displays the combined image data on the display device 136.

The combined image data is generated by combining the integration image data and the plurality of pieces of material decomposition image data in the combined region of interest at combination ratios of 0 to 1. In the present exemplary embodiment, the combination ratio of the integration image data is 0.5, and the total combination ratio of the plurality of pieces of material decomposition image data is 0.5. Among the pieces of material decomposition image data, the total combination ratio of 0.5 is distributed based on the material density ratios. The display control unit 114 may set the combination ratios of the plurality of pieces of material decomposition image data inside the combined region of interest to be higher than a predetermined value. This can highlight the material decomposition image data inside the combined region of interest. The display control unit 114 may set the combination ratios outside the combined region of interest to be lower than a predetermined value. The display device 136 illustrated in FIG. 2 displays the integration image data in grayscale, and displays the plurality of pieces of material decomposition image data in respective different colors.

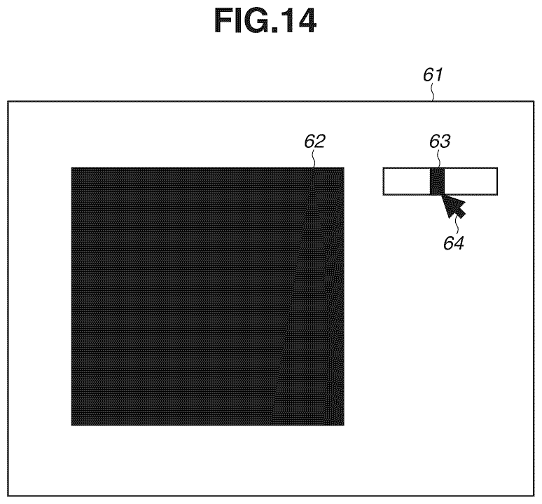

FIG. 14 is a diagram illustrating a display mode of the display device 136.

The display device 136 displays a display screen 61, an image display window 62, a combination ratio setting bar 63, and a cursor 64. Examples of the display device 136 include a display accompanying the information processing apparatus 100 and a mobile terminal of hospital staff accessed via an external server.

The combination ratios may be set by the user in advance. The user may set the combination ratios using the combination ratio setting bar 63 on the display device 136 of FIG. 14. In regions where a plurality of materials is mixed in the material decomposition image data, the display device 136 may display the combined image data with additional coloring. The display device 136 may also display information for identification of the subject (patient information such as a patient identifier [ID] and name), information about the materials in the material decomposition image data, and combination parameters related to the generation of the combined image data.

The information processing apparatus 100 according to the present exemplary embodiment can acquire regions of interest from material decomposition image data in a plurality of respective energy bands transmitted through a subject, and acquire a combined region of interest of the material decomposition image data. Moreover, the information processing apparatus 100 can store partial image data of the material decomposition image data corresponding to the combined region of interest, and integration image data. The data can thus be stored with a reduced data amount as compared to the case where the pieces of material decomposition image data in the plurality of respective energy bands are stored.

While the display control unit 114 generates and displays combined image data by combining the integration image data with the plurality of pieces of material decomposition image data, the pieces of material decomposition image data may be combined with each other. Energy band-specific X-ray image data and the material decomposition image data may be combined with each other. The images based on the detection signals can thereby combined and displayed.

### (First Modification of Second Exemplary Embodiment)

In the above-described exemplary embodiment, in step S52, the data processing unit 112 transmits the integration image data to the storage unit 113 without performing the data amount reduction processing on the integration image data. However, the present exemplary embodiment is not limited thereto, and may be configured to perform the data amount reduction processing on the integration image data. Specifically, the data processing unit 112 may generate partial image data of the integration image data outside the combined region of interest, and transmit the partial image data to the storage unit 113 instead of the entire image. As for the material decomposition image data, partial image data of the material decomposition image data inside the combined region of interest is generated and the partial image data is transmitted to the storage unit 113. In such a manner, the integration image data and the material decomposition image data with the reduced data amount can be stored. More specifically, the storage unit 113 stores the partial image data of the material decomposition image data inside the combined region of interest and the partial image data of the integration image data outside the combined region of interest.

### (Second Modification of Second Exemplary Embodiment)

In the above-described exemplary embodiment, in step S51, the region of interest acquisition unit 111 acquires regions where the material decomposition image data reaches or exceeds a threshold, as the regions of interest. However, the region of interest acquisition unit 111 may be configured to set a specific disease region or biological structure as a region of interest. To set a specific disease region or biological structure as a region of interest, an additional mask for the specific disease region or biological structure may be input. The user may specify the region of interest using the operation device 137. The region of interest acquisition unit 111 may be configured to automatically generate the mask for the specific disease region or biological structure through image recognition processing on the material decomposition image data and/or the integration image data.

The region of interest here may be a region derived from a biological structure, such as blood vessels, bones, and organs, or a specific region in the living body, such as primary cancer and metastatic cancer. The region derived from a biological structure or the specific region in the living body may be acquired based on the user's specification performed using the operation device 137. Such a region may be acquired from an apparatus or program other than the information processing apparatus 100 over a network, or generated inside the information processing apparatus 100. The region of interest can thereby be acquired based on conditions irrespective of material densities.

The description of the exemplary embodiments has been given of a case where the X-ray CT device captures images with the chest and abdomen as the subject. However, the subject may be the abdomen, head, or whole body. The subject is not limited to the human body, and may be any other thing. Examples may include animals, such as pigs and mice, and objects, such as cargoes and airport baggage.

A computer program for realizing the functions of the exemplary embodiments can be supplied to a computer via a network or a memory (not illustrated), and the computer program can be executed by a processor (not illustrated). The computer program causes a computer to execute the information processing method described above. That is, the computer program is a program that realizes the functions of the information processing apparatus by a computer. The memory stores the computer program.

According to an exemplary embodiment of the present disclosure, the data amount of image data that is stored in a storage unit of an information processing apparatus can be reduced.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

This application claims the benefit of Japanese Patent Applications No. 2023-113002, filed Jul. 10, 2023, and No. 2024-068245, filed Apr. 19, 2024, which are hereby incorporated by reference herein in their entirety.

What is claimed is:

1. An information processing apparatus comprising:
an image acquisition unit configured to acquire material decomposition image data based on detection signals related to X-ray photons transmitted through a subject in a plurality of respective energy bands;
a region of interest acquisition unit configured to acquire a region of interest in the material decomposition image data;
a data processing unit configured to generate partial image data of the material decomposition image data corresponding to the region of interest; and
a storage unit configured to store the partial image data of the material decomposition image data.

2. The information processing apparatus according to claim 1, wherein the region of interest acquisition unit is configured to acquire a region where a predetermined material is present in the material decomposition image data, as the region of interest.

3. The information processing apparatus according to claim 1,
wherein the region of interest acquisition unit is configured to set a region of interest mask for the region of interest of the material decomposition image data, and
wherein the data processing unit is configured to generate the partial image data of the material decomposition image data based on the region of interest mask.

4. The information processing apparatus according to claim 3, wherein the data processing unit is configured to set a storage region including the region of interest represented by the region of interest mask, and generate the partial image data by extracting the storage region.

5. The information processing apparatus according to claim 1, wherein the data processing unit is configured to acquire the partial image data inside the region of interest of the material decomposition image data, and delete image data in a region outside the region of interest of the material decomposition image data.

6. The information processing apparatus according to claim 1, wherein the storage unit is configured to store the partial image data corresponding to the region of interest of the material decomposition image data, and store integration image data based on the detection signals.

7. The information processing apparatus according to claim 6, wherein the storage unit is configured to store the partial image data of the material decomposition image data inside the region of interest, and store partial image data of the integration image data outside the region of interest.

8. The information processing apparatus according to claim 1,
wherein the image acquisition unit is configured to acquire a plurality of pieces of material decomposition image data,
wherein the region of interest acquisition unit is configured to acquire a region of interest in each of the plurality of pieces of material decomposition image data, and
wherein the storage unit is configured to store partial image data corresponding to the region of interest in each of the plurality of pieces of material decomposition image data.

9. The information processing apparatus according to claim 8, wherein the region of interest acquisition unit is configured to acquire regions where respective materials are present in the plurality of pieces of material decomposition image data, as the regions of interest.

10. The information processing apparatus according to claim 9, wherein the region of interest acquisition unit is configured to set a threshold about a predetermined physical quantity for each of the pieces of material decomposition image data, and acquire a region where the physical quantity is higher than or equal to the threshold, as the region of interest.

11. The information processing apparatus according to claim 8,
wherein the region of interest acquisition unit is configured to set a region of interest mask for the region of interest in each of the pieces of material decomposition image data, and
wherein the data processing unit is configured to generate the partial image data of each of the pieces of material decomposition image data based on the region of interest mask.

12. The information processing apparatus according to claim 1,
wherein the region of interest acquisition unit is configured to acquire a region of interest in combined image data obtained by combining a plurality of pieces of material decomposition image data, and set a combined region of interest mask,
wherein the data processing unit is configured to generate partial image data of the combined image data, based on the combined region of interest mask, and
wherein the storage unit is configured to store the partial image data of the combined image data.

13. The information processing apparatus according to claim 12, wherein the data processing unit is configured to set a storage region including the combined region of interest mask, and generate the partial image data of the combined image data based on the storage region.

14. The information processing apparatus according to claim 1, wherein the storage unit is configured to store the partial image data of the material decomposition image data on a material basis.

15. An information processing apparatus comprising:
an image acquisition unit configured to acquire a plurality of pieces of material decomposition image data based on detection signals related to X-ray photons transmitted through a subject in a plurality of respective energy bands;
a selection unit configured to select a material to be identified; and
a storage unit configured to store at least one piece of material decomposition image data from among the plurality of pieces of material decomposition image data, based on the selected material to be identified.

16. An information processing apparatus comprising:
an image acquisition unit configured to acquire a plurality of pieces of material decomposition image data generated from X-ray image data in a plurality of energy bands based on detection signals related to X-ray photons transmitted through a subject in the plurality of respective energy bands;
a selection unit configured to select a material to be identified; and
a storage unit configured to store energy band-specific X-ray image data from which material decomposition image data related to the selected material to be identified is generated.

17. An information processing method comprising:
acquiring material decomposition image data based on detection signals related to X-ray photons transmitted through a subject in a plurality of respective energy bands;
acquiring a region of interest in the material decomposition image data;
generating partial image data of the material decomposition image data corresponding to the region of interest; and
storing the partial image data of the material decomposition image data.

18. A non-transitory computer-readable storage medium storing a program that causes a computer to perform the information processing method according to claim 17.
